(19) United States
(12) Patent Application Publication
(10) Pub. No.: US 2022/0398942 A1
(43) Pub. Date: Dec. 15, 2022

(54) METHOD FOR MANUFACTURING A TRIDIMENSIONNAL BLOOD VESSEL

(71) Applicants: INSERM (Institut National de la Santé et de la Recherche Médicale), Paris (FR); Ecole Nationale Superieure d'Arts et Metiers (ENSAM), Paris (FR); Etablissement Français du Sang, La Plaine Saint Denis (FR); Assistance Publique Hôpitaux de Paris, Paris (FR); Université Paris Est Creteil Val de Marne, Creteil (FR); Fondation Imagine, Paris (FR)

(72) Inventors: Pablo BARTOLUCCI, Creteil (FR); Kim-Anh NGUYEN-PEYRE, Creteil (FR); Christian KASSASSEYA, Creteil (FR); Margaux RITTER, Paris (FR); Frédéric SEGONDS, Paris (FR); Pierre VEDEL, Paris (FR)

(21) Appl. No.: 17/775,087

(22) PCT Filed: Nov. 5, 2020
(86) PCT No.: PCT/EP2020/081041

§ 371 Date: May 6, 2022

(30) Foreign Application Priority Data
Nov. 6, 2019 (EP) 19306437.5
Dec. 6, 2019 (EP) 19306595.0

(51) Int. Cl. G09B 23/30; C12N 5/071; G06T 19/20; G06T 7/00; B29C 64/124

(52) U.S. Cl. CPC G09B 23/306; G09B 23/303; C12N 5/0691; G06T 19/20; G06T 7/0012; B29C 64/124; G06T 2219/2021; G06T 2207/10088; G06T 2207/30101

(57) ABSTRACT

Method for manufacturing a tridimensional blood vessel model using stereolithography and optionally cell culture. Applications include surgery training, research on pathology such as SCDs and in vitro drug testing e.g. for antiplatelets. Existing models are not compatible with cell culture and cannot withstand high pressure, as opposed to the present invention. Stereolithography allows modelling of complex vessels such as carotid siphons as opposed to other existing methods.

The present invention relates to the field of artificial blood vessel manufacturing. Artificial blood vessels can be used for surgeon training, for drug testing in vitro or for prosthesis, among other applications.

In order to better understand pathologies such as SCDs, to allow in vitro drug testing and surgeon training, there is a need for an accurate tridimensional blood vessel model.

Indeed, in order to test drugs such as antiplatelets, there is no in vitro model available which lead to direct in vivo testing.

A few models are available for surgeon training which reproduce the general shape and mechanical properties of a given vessel. However, none of these comprise actual vessel cells allowing e.g. evaluating potential damage on the vessel walls. Furthermore, known models cannot handle a high blood flow, if any, thus preventing a proper simulation of the surgery.

Such models are difficult to build, especially for blood vessels presenting a complex geometry such as carotid siphons.

There is thus a need for a tridimensional blood vessel model which can effectively simulate in vivo conditions by withstanding high blood flow and by allowing proper vessel cell growth.

An object of the present invention is therefore to provide a method for manufacturing a tridimensional blood vessel model comprising:

i) providing medical imaging data of a blood vessel,

ii) generating a mesh out of said medical imaging data,

iii) generating a tridimensional blood vessel model out of said mesh through stereolithography.

The mesh size is preferably chosen as accurately as possible given the stereolithography printer's resolution, as it has a high impact on the accuracy of the model.

The method according to the invention preferably further comprises submitting the blood vessel model to a fluid treatment such as plasma or whole blood treatment so as to provide the tridimensional blood vessel model with a ligand-compatible inside wall. Indeed, most materials compatible with stereolithography are hydrophobic, which tends to hamper the adherence of other layers inside the models, especially blood vessel tissue.

The method then preferably further comprises providing a layer of ligand on the ligand-compatible inside-wall. The ligands can serve as binders for other layers such as blood vessel tissue.

Preferably, the method then further comprises growing blood vessel cells on the ligand-compatible inside wall, preferably human umbilical vein endothelial cells and/or smooth muscle cells. cell-culture on the inside walls of the model is a major feature which allow mimicking an actual blood vessel.

The method according to the present invention preferably further comprises modifying the medical imaging data of step i or the mesh of step ii so that the model generated in step iii comprises a pathology which was not present in the provided medical imaging data.

The pathology is preferably chosen among aneurism and steno sis.

Preferably, the provided medical imaging data comprise MRI data although other medical imaging data can be used, especially Doppler, computerized tomography (CT) scan, or arteriography imaging data.

Typically, the blood vessel is a carotid, a coronary artery, the aorta, an artery of the lower or upper limbs. Preferably, the blood vessel is a carotid. Indeed, the structure of the carotid is very complex and the method according to the invention allows circumventing geometric issues thanks to stereolithography, which cannot be solved with other methods. Indeed, for most 3D printing methods there is a need to include a support inside the blood vessel cavity, the lumen, so as to prevent the whole model to collapse during its manufacturing. Such support is then very complicated to remove in case of a complex structure such as the carotid siphon.

Another object of the present invention is a tridimensional blood vessel model presenting a cavity, wherein the model comprises a polymeric skeleton, endothelial cells, preferably human umbilical vein endothelial cells, and smooth muscle cells.

Such model is preferably obtained by a method according to the present invention.

Preferably, the polymeric skeleton comprises a polymer which is compatible with stereolithography. Preferably, the polymeric skeleton comprises the material selected from the group consisting of the material commercialized under the denomination TuskXC2700T by the company Materialize; the material commercialized under the denomination Polyl500 by the company Materialize; the material commercialized under the denomination ProtenGen White by the company Materialize; the material commercialized under the denomination Tusk Somos SolidGrey3000 by the company Materialize; the material commercialized under the denomination TuskXC2700W by the company Materialize; the material commercialized under the denomination Taurus by the company Materialize; the material commercialized under the denomination Xtrem by the company

Materialize; the material commercialized under the denomination NeXt by the company Materialize; the material commercialized under the denomination PerFORM by the company Materialize. More preferably, the polymeric skeleton comprises the material commercialized under the denomination TuskXC2700T by the company Materialize. The material is preferably transparent so as to ease the observation inside the model, compatible with high flows, and compatible with cell culture. The model according to the present invention can preferably withstand flows higher than 500 mL per minute.

The tridimensional blood vessel can further comprise connectors configured to enable and/or control a fluid circulation into and out of the model. Indeed, such connectors allow performing fluidic experiments so as to study the shear stress of the carotid walls, or the effect of a drug such as antiplatelets, vasodilators, anti-inflammatory drugs, biotherapies, or the effect of intravascular or surgical devices.

Another object of the present invention is a computer program product comprising code configured to, when executed by a processor or an electronic control unit, perform the method according to the present invention. Indeed, performing the method by a computer program product can allow the generation of personalized carotid models which can prove useful e.g. for surgery training.

Another object of the present invention is a utilization of a tridimensional blood vessel model, preferably of a tridimensional carotid model, for in vitro drug testing.

The invention can be better understood at the reading of the detailed examples below, which constitute non-limitative embodiments of the present invention and at the examining of the annexed drawing, on which:

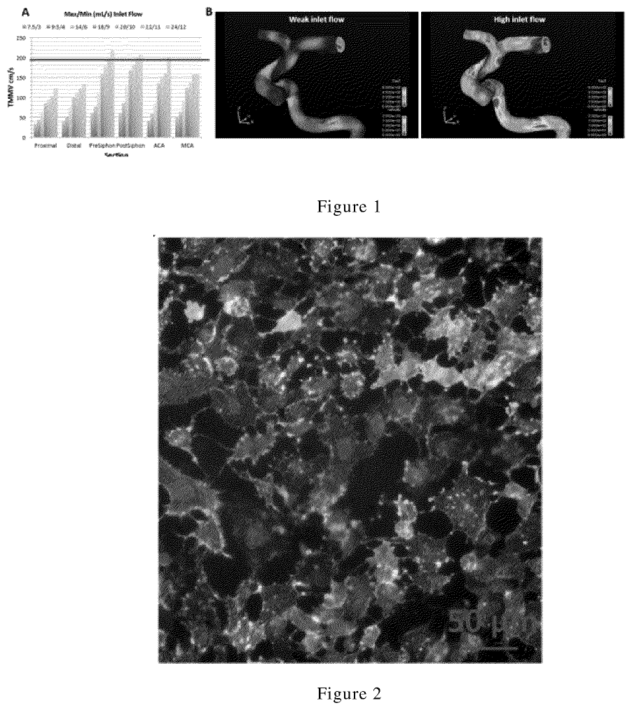

FIG. 1 shows the result from flow simulation on a model according to the present invention;

FIG. 2 shows HUVECs culture on a carotid model wall;

FIG. 3 is a photography of a perfusion assay at high inlet flow by a programmable pump on a model according to the invention;

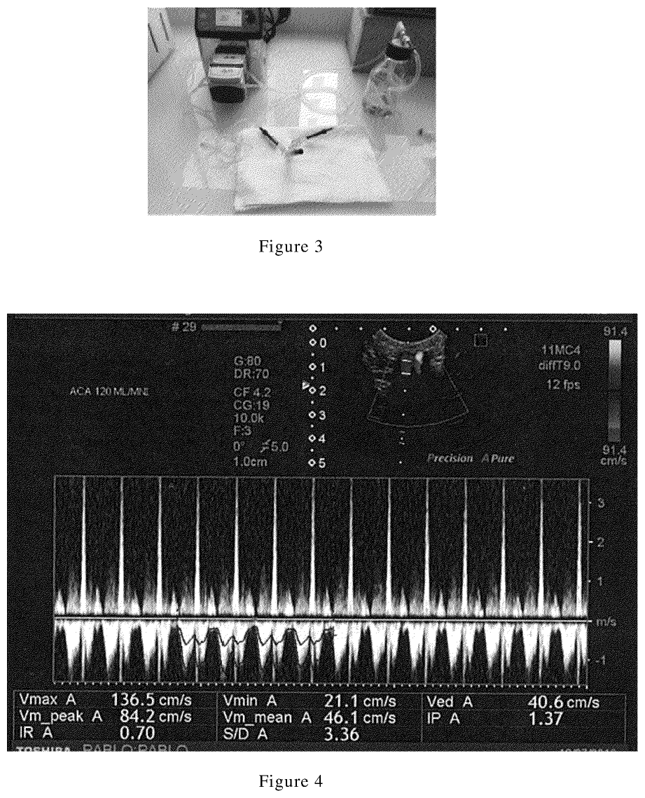

FIG. 4 shows preliminary results of ultrasonography in carotid model;

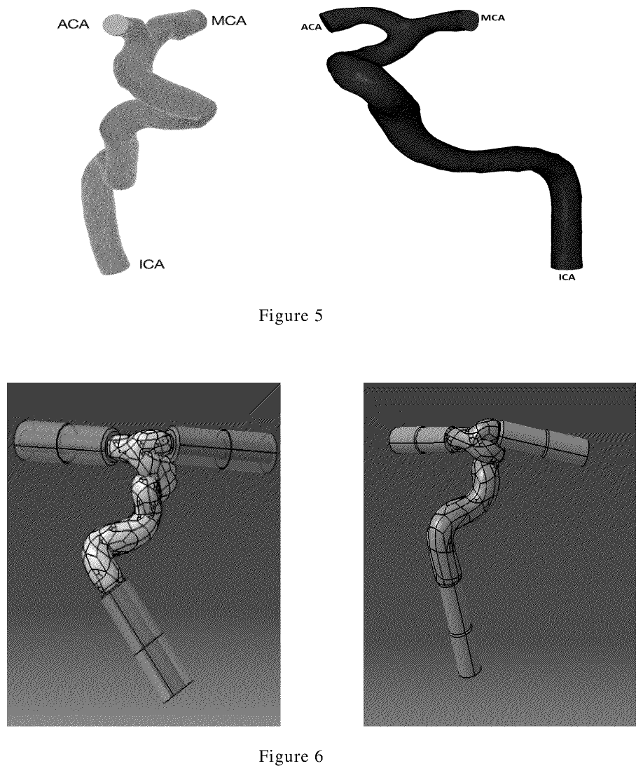

FIG. 5 show MRA data of child and adult carotids;

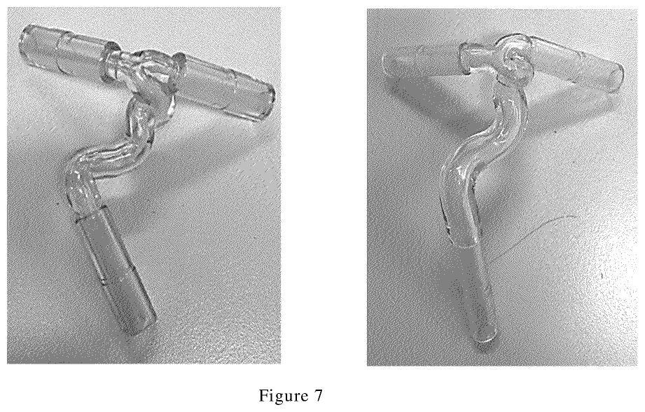

FIG. 6 shows meshes generated out of the data of FIG. 5; and

FIG. 7 is a photography of 3D-printed models according to the invention obtained out of the meshes of FIG. 6.

It is understood that the described embodiments are not restrictive and that it is possible to make improvements to the invention without leaving the framework thereof.

Sickle cell disease (SCD) is the most prevalent and severe monogenic disorder due to a mutation in the b-globin gene, responsive for a pathological haemoglobin (HbS) which polymerize in hypoxia condition.

Strokes in SCD patients are related to the appearance during childhood of stenosis on carotid and cerebral anterior and middle arteries by hemodynamic and/or embolic mechanisms. Despite the reduction in the risk of stroke by chronic blood exchange transfusion (BET) to prevent the cerebral vasculopathy (CV), the cumulative risk is 22.6% for stenosis and 37.1% for silent stroke by age 14 years. While CV develops during childhood, the risk of complications remains lifelong, with a greater stroke recurrence in adulthood on our cohort study. To date, allogenic hematopoietic stem cell transplant (HSCT) offers the only potential cure for SCD but is limited by the number of potential HLA-matched HSC donors, leading to consider gene therapy as the most promising curative treatment. When bone marrow allograft cannot be performed, SCD conventional treatment aims at decreasing HbS percentage by BETs in patients deemed high risk. However, BETs are limited by alloimmunization and iron overload, and some patients are not responders. When the CV is not severe hydroxyurea (HU) could replace BET.

Despite the use of these conventional treatments for years in the management of SCD patients and the development of new therapeutic approaches by HSCT or gene therapy, the prediction and assessment of their effects and efficacy are impaired by our only partial understanding of the mechanisms leading the development of CV and the lack of specificity of imaging techniques or biomarkers at the individual level. The critical consequences are the yet poor detection of patients at risk, and the poor prediction of treatment effects and efficacy that may complicate the choice of the most appropriate therapeutic option to be proposed to each patient, depending on his/her age and condition.

Therefore, by developing a 3D carotid model reproducing exactly vascular parameters of a SCD patient the aim is to: (i) determine the mechanism of CV development in SCD, (ii) evaluate or predict the efficacy of treatments by implementing innovative tools and (iii) treat SCD patients by an innovative therapeutic strategy. Furthermore, this work can be transposed to many other pathologies and domains such as vessel prothesis in cardiovascular diseases, neurology and surgery training.

Based on magnetic resonance angiography (MRA) data (FIG. 5: left corresponds to a child carotid whereas right corresponds to adult carotid) and hemorheological conditions of each patient, internal carotids until middle and anterior cerebral arteries were modelled (FIG. 6: left corresponds to a child data and right to an adult data). Three dimensional simulations of the Navier-Stokes equations are performed in patient specific geometries, including the state-of-the-art techniques of Computational Hemodynamics (backflow stabilization, multiscale coupling and uncertainty quantification) and other factors (such as the increase of the ejection fraction, or the drop of peripheral resistances). Blood viscosity was based on our SCD cohorts. Flow velocities (TMMV), wall shear stress (WSS) and shear rates in different areas of modelled carotid were then measured according to flow variations.

Modelled carotid was printed by stereolithography technique (FIG. 7 shows a child carotid model on the left and an adult carotid model on the right) according to computer design (CATIA software). Then Doppler parameters from patients will be imported in a programmable pump (OB1-Elvesys Inc) for flow assays with blood mimicking fluid to measure TMMV and WSS at different areas in carotid. Incorporation of resting or activated platelets in BMF will allow to evaluate impact of high WSS on platelets degranulation.

A flow co-culture of smooth muscle cells (SMCs) and endothelial cells, preferably human umbilical vein endothelial cells (HUVECs) on carotid wall is developed in parallel. Flow experiments will be performed according to pathophysiological conditions identified in our previous SCD cohorts or individually in patients (before and after receiving treatment) including hemolysis (lysis red blood cells or purified haemoglobin), different percentage of therapeutic haemoglobin, viscosity and inlet flow measures. Finally, HUVECs and SMCs at different zones of the carotid undergoing high/low WSS and oscillatory flow will be analysed for following aspects: (i) endothelial damage and dysfunction markers; (ii) the transcriptomic, proteomic and secretion profile of HUVECs and (iii) the proliferation and secretion profile of SMCs.

On one hand, by modification of input conditions and evaluation of output data, our 3D personalized model can enable the physiopathology of CV and allow to predict their evolution according to received treatment. This innovative model is a pertinent tool to evaluate individually effectiveness of new therapeutic strategies in SCD patients. On other hand, with a similar process, it is possible to manufacture customized and complexes vessels for all kind of in vitro studies, prothesis domain and surgery training.

Stereolithography is used to print the 3D carotid models. Stereolithography is one of the most important additive manufacturing technologies currently available. 3D SLA printers belong to a family of additive manufacturing technologies called photopolymerization in tanks. This technology involves curing or solidifying a light-reactive thermoset material known as “resins”. It is a liquid photosensitive polymer that hardens with a radiating light source, which provides the energy needed to induce a chemical reaction, binding a large number of small molecules and forming a highly crosslinked polymer.

This technique was chosen because it meets all the needed criteria. The conditions are as follows: the material must be transparent to allow observation of cells, strong enough to pass important flows in the carotid and finally the support need to be outside the geometry. The support is the part that allows the maintenance of the part correctly in the tank. Then it is removed at the end of printing.

The process is that the piece is built layer by layer. At each layer the laser solidifies the resin necessary for the construction of the part. Then the tray goes down and so on until the piece is finished. The model is cleaned by removing the excess of resin, rinsing with water and finally rinsing with ethyl alcohol, before removing the supports.

The material used is TuskXC2700T. It's the world's first stereolithography material that combines high stiffness with superior impact resistance. This transparent material is sold by Materialise. Its shore hardness is about 81D.

The 3D carotid model is to be as close to the arterial wall histology as possible and to allow the reproduction of various pathological conditions observed in SCD patients then to evaluate histological, morphological and functional aspects of arterial wall (intima, endothelial cells and sub-endothelial proteins) and the degranulation of platelets.

To this end, a first step consists in developing a 2D static culture of SMCs differentiated from adipose tissue-derived stem cells (ASCs) on carotid wall under hypoxia condition. This differentiation is validated by flow cytometry, immunofluorescence staining and Western Blot to detect the expression of smooth muscle cells-specific markers, including early marker smooth muscle alpha actin, middle markers calponin, caldesmon, and lte marker smooth muscle myosin heavy chain.

The 2D co-culture with HUVECs on carotid wall is performed in a second phase. This co-culture is optimized and characterized by techniques described previously as well as histological studies to evaluate the phenotype, the morphology, the proliferation and the viability of HUVECs and SMCs.

In a subsequent step, the 3D flow co-culture of HUVECs and SMCs is developed in 3D printing carotid. This phase aims to reproduce the blood arterial wall histology parameters and is validated by using especially histological analyses.

In previous 3D co-culture of SMCs and HUVECs flow experiments are performed in blood mimicking fluid (BMF) according to pathophysiological conditions identified in cohort studies or individually in patients treated with gene therapy. Particularly hemolysis (hemolysate or free purified Hb), viscosity and vascular flow measures are applied in the 3D printing model thanks to the programmable pump importing Doppler measures (OB1-Elver flow Inc). Then ECs and SMCs are analyzed at different zones of the carotid undergoing high/low wall shear stress and oscillatory flow (dissection of interest areas) for following aspects

(i) The ECs responses:
actin network reorganization, permeability, endothelial injury and endothelial barrier dysfunction
Platelets and RBCs adhesion
Oxidative stress, inflammation and activation markers
Transcriptomic, proteomic and secretion profile of HUVECs
Detection of Apoptosis, necrosis and Viability: Increases in mitochondrial membrane potential lead to increased mitochondrial membrane permeability and the release of soluble proteins such as cytochrome c and pro-caspases.

(ii) The proliferation and secretion profile of SMCs

(iii) The interaction ECs-SMCs

(iv) The endothelial response is also conditioned by genetic polymorphisms involving inflammatory, proliferative or vaso-reactive signalling pathways. The model can also incorporate an invalidation of genes of interest (by using SiRNA/ShRNA) to study their role in development of cerebral vasculopathy.

In parallel, apheresis platelet concentrates (APC) are reconstituted in BMF at physiologic concentration before perfusing in 3D printing arteries allowing to evaluate the impact of shear rate on platelets responses and degranulation in carotid model.

Following interaction with endothelial cell as all platelets constitutively express CD41a; this marker was used to define gates in subsequent experiments. Activated platelets are characterized by their expression—among other markers—of CD62P, PAC-1 and CD63 sCD62P, sCD40L, Gro Alpha, HMGB1, CXCL12, CXCL14, TSLP, PF4, RANTES and Serotonin content in platelet supernatants were quantified using commercial Luminex or ELISA kits. The quantification of intracellular proteins from the platelets was performed using ELISA technology (Phospho-Akt, and PKC).

Depolarization of mitochondria results in a decrease in the mean fluorescence intensity of platelet-bound DiOC6(3) which allows measuring platelet mitochondrial membrane potential (ΔΨm), following interaction with endothelial cells. To assess platelet phosphatidylserine externalization, phosphatidylserine exposed in the membrane of apoptotic or necrotic cells was quantified by flow cytometry.

In order to determine platelet aggregation, the aggregometer was calibrated by using PRP and platelet poor plasma. Platelet aggregation was monitored by the Thrombo-aggregometer. At the end of platelet stimulation, ADP is added to each PRP suspension already containing test items to confirm that platelets are still responsive to stimulation.

Preliminary results in intra vascular hemolysis model showed that platelet thrombus can be rapidly formed in a GPIIbIIIa dependent at sub endothelial exposure areas. An hypothesis is that platelet thrombus could be formed in our 3D printing model due to high shear stress and hemolysis. Platelet thrombus formation can be evaluated on artery wall and assessed by anti GPIIbIIIa treatment.

An hypothesis is that the platelets soluble products including inflammatory and fibrosis factors in particular TGFb and Serotonin having a fibrosating and proliferating effect on the arterial wall, which could induce a vascular dementia and spread inflammation of ECs in the whole system. Furthermore, the secretion resulted from mast cells may also be involved in physiopathology of cerebral vasculopathy.

Washed platelets are exposed to various shear stresses (low, high, low to high and high to low) in a cone-plate viscometer. Platelets degranulation products can then be infused with BMF in 3D printing arteries model.

In a subsequent stage of study, these fluidic experiments contribute to improve the estimation of flow velocities in inaccessible zones by Tc Doppler allowing the amelioration of hemorheological parameters in patients. The 3D carotid model is also very helpful for testing the innovative therapies and molecules.

Flow simulations were performed in real geometries of the cerebral arteries of several different patients (children and adults) to first study the correlation between fluid mechanical properties and stenosis location. The results suggest that the carotid inlet flow is responsible for the pathological intra cranial velocities. Moreover, the geometry and the range of flows in adults were not able to the reach the pathological intracranial velocity threshold. This result showed for the first time an explanation of the difference in intracranial velocity between children and adults (FIG. 1A). At high carotid inlet flow, areas of high and low wall shear stress (WSS) appeared in children, while low and homogenous WSS was observed at weak inlet flow (FIG. 1B), suggesting the existence of turbulent flow that could lead to arterial wall damages.

Additive Manufacturing method has been chosen. All the 3D models for 3D printing arteries shown on FIG. 6 are designed by CATIA software (Dassault System). A carotid was 3D printed reproducing the exact SCD child's one. The material of artificial carotid is compatible with HUVECs culture (FIG. 2) and fluidic experiment at high inlet flow (FIG. 3). On Doppler ultrasonography, the velocities measured in different sections of carotid were comparable to patient's data and these velocities were modified according to variations of inlet flow values (FIG. 4).

Preliminary results from mathematical modelling show that geometry and velocities of arteries have a major pathophysiological effect, making animal models inappropriate. Hence the need to confirm and complete the mathematical modelling results by an in vitro model as per the present invention which allows fabricating the internal carotids reproducing the exact patient's ones including anatomy and hemodynamic parameters in a way compatible with 3D flow cells culture and fluidic experiment at high inlet flow.

Many requirement specifications were challenged and resolved:
Material used must to be transparent, compatible for cell culture, resistant to high flow and compatible for 3D printing technologies.
Manufacturing method: Additive manufacturing (AM) has been chosen because of the complexity of carotid structure. According to previous works, the main role of AM technologies is to produce parts and devices that are geometrically complex, have graded material compositions, and can be customized. The conventional 3D printing technologies such as Fused Desposition Modeling (FDM) or Material Jetting (Polyjet technology from Stratasys) was not compatible. Moulding technique was tested, but it leads to a lumen which was rough and would modify the wall shear stress in fluidic experiments. The stereolithography was chosen and showed easiness to remove outside support material. Moreover, the parameters of printed carotids were identical to CAD (computer aided design) model.
Surface treatment for cell culture: Material surface is mostly hydrophobic which requires a plasma treatment

The present invention enables the first attempt to understand the physiopathology of cerebral vasculopathy in sickle cell diseases. The mathematical modeling of vascular blood flow complemented by in vitro flow experiments using innovative 3D-printing carotids from MRI and Doppler data will allow a better understanding of the determinants of arterial velocities. This will inform us to improve the methods of cerebrovascular assessment in patients with SCD and to greatly improve the follow-up of innovative treatments at the individual level.

Its originality lies in its global approach to reconstruct physio-pathological conditions in an experimental model.

Therefore, this project is developed to determine the mechanism of development of diseases that affect the arteries such as atherosclerosis, atheroma, arterial fibromuscular dysplasia or the mechanism of development of cerebral vasculopathy in sickle cell disease and to predict the efficacy of new treatments as gene therapy and allograft, or the efficacy of intravascular or surgical devices. There are 3 steps in this project: the first step is mathematical modelling of internal carotid, which demonstrated that the carotid inlet flow is responsible for the pathological intra cranial velocities. These accelerations are generally followed by the appearance of arterial lesions detected by magnetic resonance angiography (MRA) suggesting the carotid inlet flow could be responsible for the appearance of a steno sis. Moreover, the mathematical modelling showed also that geometry and velocities of arteries have a major pathophysiological effect, thus, making animal models inappropriate. Therefore, the manufacturing of artificial internal carotid in second step is necessary to confirm result of step 1. And finally, in the artificial carotid model, the last step of this project allows to investigate the impacts of carotid inlet flow on carotid wall damages and on platelets degranulation.

It is understood that the described embodiments are not restrictive and that it is possible to make improvements to the invention without leaving the framework thereof.

Unless otherwise specified, the word “or” is equivalent to “and/or”. Similarly, the word ‘one’ is equivalent to ‘at least one’ unless the contrary is specified. Unless otherwise specified, all percentages are weight percentages.

1. Method for manufacturing a tridimensional blood vessel model comprising:
providing medical imaging data of a blood vessel,
generating a mesh based on said medical imaging data, and
generating a tridimensional blood vessel model based on said mesh through stereolithography.

2. The method of claim 1, further comprising submitting the blood vessel model to a fluid treatment thus providing the tridimensional blood vessel model with a ligand-compatible inside wall.

3. The method of claim 2, further comprising providing a layer of ligand on the ligand-compatible inside-wall.

4. The method of claim 3, further comprising growing blood vessel cells on the ligand-compatible inside wall.

5. The method of claim 1, further comprising modifying the medical imaging data or the mesh so that the tridimensional blood vessel model comprises a pathology which was not present in the medical imaging data.

6. The method of claim 5, wherein the pathology is aneurism or stenosis.

7. The method of claim 1, wherein the medical imaging data comprise MRI data.

8. The method of claim 1, wherein the blood vessel is a carotid.

9. A tridimensional blood vessel model obtained by
providing medical imaging data of a blood vessel,
generating a mesh based on said medical imaging data, and
generating a tridimensional blood vessel model based on said mesh through stereolithography.

10. A tridimensional blood vessel model comprising a cavity, wherein the tridimensional blood vessel model comprises a polymeric skeleton and endothelial cells.

11. The tridimensional of claim 10, wherein the polymeric skeleton comprises a polymer which is compatible with stereolithography.

12. The tridimensional of claim 10, wherein the polymeric skeleton comprises the material commercialized under the denomination TuskXC2700T by the company Materialize.

13. The tridimensional of claim 10 further comprising connectors configured to enable and/or control a fluid circulation into and out of the model.

14. A computer program product comprising code configured to, when executed by a processor or an electronic control unit,
provide medical imaging data of a blood vessel,
generate a mesh based on said medical imaging data, and
generate a tridimensional blood vessel model based on said mesh through stereolithography.

15. An in vitro drug testing method comprising using a tridimensional blood vessel model.

16. The method of claim 2, further comprising modifying the medical imaging data or the mesh so that the model comprises a pathology which was not present in the medical imaging data.

17. The method of claim 3, further comprising modifying the medical imaging data or the mesh so that the model comprises a pathology which was not present in the medical imaging data.

18. The method of claim 4, further comprising modifying the medical imaging data or the mesh so that the model comprises a pathology which was not present in the medical imaging data.

19. The method of claim 4, wherein the blood vessel cells are at least one of human umbilical vein endothelial cells and smooth muscle cells.

20. The tridimensional blood vessel model of claim 10, wherein the endothelial cells are at least one of human umbilical vein endothelial cells and smooth muscle cells.
